(19) United States
(12) Patent Application Publication
(10) Pub. No.: US 2026/0124044 A1
(43) Pub. Date: May 7, 2026

(54) SPINAL IMPLANT HAVING COMPLIANT SURFACE

(71) Applicant: Integrity Intellect Inc., Jupiter, FL (US)

(72) Inventors: Christopher Walsh, Jupiter, FL (US); Brian A. O'Shaughnessy, Nashville, TN (US)

(21) Appl. No.: 19/375,974

(22) Filed: Oct. 31, 2025

Related U.S. Application Data
(60) Provisional application No. 63/716,062, filed on Nov. 4, 2024.

(51) Int. Cl. A61F 2/44; A61F 2/30; A61L 27/06; A61L 27/18

(52) U.S. Cl. CPC A61F 2/447; A61F 2/30767; A61L 27/06; A61L 27/18; A61F 2002/30116; A61F 2002/30153; A61F 2002/30261; A61F 2002/30622

(57) ABSTRACT

The invention involves an interbody implant (spacer) and a method of manufacturing an interbody implant for use in spinal procedures, having one or more compliant surfaces to more evenly distribute the load applied to the bone while in use, as well as aid in bone growth and attachment. The interbody spacer has one or more unique surfaces designed to comply (flex) upon receiving a predetermined load. By flexing, the surface contact area between the implant and the bone is increased to distribute the load over a larger area. In order to provide the flexing, at least a portion of the implant is constructed from a plurality of bar springs that are interconnected or integrally connected with each other to conform to a surface while providing sufficient support for use as an implant.

### RELATED APPLICATIONS

In accordance with 37 C.F.R 1.76, a claim of priority is included in an Application Data Sheet filed concurrently herewith. Accordingly, under 35 U.S.C. §119(e), the present invention claims priority of U.S. Patent Application No. 63/716,062, entitled “SPINAL IMPLANT HAVING COMPLIANT SURFACE”, filed on November 4, 2024. The contents of the above referenced application are herein incorporated by reference in its entirety.

### FIELD OF INVENTION

The present invention relates to bone fixation devices and procedures for the placement of these devices in an individual. More particularly, the present invention relates to a device for use in spinal fusion having a compliant structure to provide more even force loading to the surrounding bone structure.

### BACKGROUND INFORMATION

Medical procedures often require the use of surgical hardware. In spine related surgeries, a common type of surgical hardware used by surgeons is an interbody implant. Typical interbody implants are constructed from very rigid materials, e.g. plastics, having the compressive strength of steel or titanium alloys, also having extremely high compression ratings. Thus, the implants are rigid and inflexible under the normal loads exerted on the bones of an in-vivo human or other animal. The implants are inserted into various portions or anatomical features of the spine, including in the cervical, thoracic, lumbar, sacroiliac joints, and facets. The implants are generally constructed to cover as much bone surface as is possible with a particular procedure for distribution of the load applied to the bone. Thus, different procedures have been developed in an attempt to provide the most stability to the implant and reduce damage to soft tissue during the procedure. For example, the degeneration of the intervertebral disk, in particular, the degeneration of the nucleus pulposus, results in a loss of height in the affected disk space which is associated with a weakening of the annulus fibrosus and of the ligaments. As a consequence, the spinal column becomes instable and is more susceptible to horizontal displacement of the vertebral bodies with respect to one another. This horizontal or vertical movement of vertebral bodies results in impairments of the nerve roots in this region and/or of the spinal marrow, with pain resulting therefrom.

The principle treatment of these symptoms consists of the surgical removal of the nucleus pulposus and the insertion of support bodies in order to restore the normal height of the disk space. While there are a number of traditional systems and methods for inserting support bodies, there are a variety of demands on both the surgeon performing an intervertebral disk procedure and on the spinal spacers themselves.

A Transforaminal Lumbar Interbody Fusion (TLIF) is a surgical procedure that uses a posterior and lateral approach to access the disc space and insert a spacer. To gain access to the disc space, typically, a facet joint is removed and access is gained via the nerve foramen. While more technically demanding of the surgeon than other fusion techniques, a TLIF offers a number of clinical advantages.

When compared to a Posterolateral Fusion (PLF), a TLIF approach leaves much more of the soft tissue intact, which is less traumatic for the patient. Further, a PLF does not provide access to the disc space.

While a Posterolateral Interbody Fusion (PLIF) provides access to the disc space, a TLIF approach also provides access to the interbody space, but without the need for manipulation of neural elements, reducing the risk of post-operative neural deficit. Additionally, in a TLIF, only a single spacer is placed. More specifically, the TLIF spacer is placed in the anterior aspect of the disc space, thus providing space for a substantial fusion mass in the posterior aspect of the disc space where the natural compression occurs.

An OLIF (oblique lateral interbody fusion) procedure minimizes the cutting of muscles and uses a single port to access the disc space and fill the interbody implant with bone material to fuse the bones.

An XLIF (extreme lateral interbody fusion) is a minimally invasive procedure using an approach from the side of the lower back. This procedure is also referred to as LLIF (lateral lumbar interbody fusion) and DLIF (direct lateral interbody fusion. XLIF does not require entry through sensitive back muscles, bones or ligaments. This typically results in less pain after surgery.

An ALIF (anterior lumbar interbody fusion) approach has several advantages but involves a major abdominal incision. The technique allows direct view of the disc space and vertebral bodies which permits easier cleaning of the disc space. In addition, the back and lateral muscles are spared from damage during the procedure, reducing postoperative pain.

However, all of these procedures suffer from a similar shortcoming. In particular, the surfaces of the bone where the implant(s) is/are placed are often not flat and may contain high or low areas. Because the implants are rigid and non-compliant, the bone adjacent the implant may be subjected to increased loads for a given area, e.g. stress risers. In addition, the bone may be pushed in front of the implant as it is inserted, also resulting in uneven surfaces and thus small contact areas between the bone and the implant, causing high loads on the bone for a given area. Other causes of end plate subsidence may include reduced bone density due to age, disease or sex. The increased loads, in some cases, cause the bone to crack or, in other cases, the bone may fail entirely under the load, requiring surgical revision of the procedure. This condition is typically referred to as endplate subsidence. The revision of this issue requires additional surgery and may require removal of additional bone material and the addition of more implant hardware to revise the failed bone. This outcome may be exacerbated when the procedures are performed on osteoporotic women where the bone is brittle and/or weakened from the condition. Due to the position of the bone surfaces and the lack of a direct view to the surgeon, preparing the bone surface to be free of surface defects is extremely difficult. In other cases, the two endplates of the vertebrae may not be parallel with respect to each other. While there are implants constructed for this specific purpose, an exact match of the opposing angled surfaces is also extremely difficult. This condition also causes increased loading to the endplates. The increased loads can cause bone failure and require additional surgery for correction. Thus, what is needed in the art is an interbody implant that includes a compliant surface which is capable of at least partially conforming to the surface of the bone which it contacts to more evenly distribute the loads applied to the bone(s).

Thus, the present invention provides an interbody implant that includes a contacting surface that is capable of at least partial compliance to match the bone surfaces. The present invention may also provide an interbody implant wherein the entire implant is constructed and arranged to include controlled compliance or flexibility to provide better load distribution to the bone’s surfaces adjacent the implant. The implant is applicable to at least the various types of interbody fusion listed above.

### SUMMARY OF THE INVENTION

Briefly, the present invention is directed towards a device, preferably an interbody implant (spacer) for use in spinal procedures, having one or more compliant surface(s) designed to more evenly distribute the load applied to the bone while in use, as well as aid in bone growth and attachment. The interbody implant is preferably designed for use as an intervertebral spacer in spinal fusion surgery, where portions of an affected disc are removed from between two adjacent vertebrae and replaced with an interbody implant that provides segmental stability, may correct a deformity, and allows for bone to grow between the two vertebrae to bridge the gap created by disk removal. The interbody spacer has one or more unique surfaces designed to comply (flex) upon receiving a predetermined load. By conforming or flexing, the surface contact area between the implant and the bone is increased to distribute the load over a larger area reducing the load particularly for the stress riser areas. In order to provide the flexing, at least a portion, and in cases the entire implant, is constructed from a plurality of bar springs that are interconnected or integrally connected with each other to conform to a surface while providing sufficient support for use as an implant. In a most-preferred embodiment, the bar springs are printed using titanium, PEEK or PEAK, or other printable material suitable for producing a spring temper. In alternative embodiments, the implants may be constructed by machining or injection molding processes and may be subsequently heat treated as needed to produce the spring temper in the material used to construct the implant. The spring bars are interconnected together in a manner to control the amount of conformance the surface of the implant may provide. The conformance allows more bone to contact the implant, thus dispersing the load over an increased area and reducing the possibility of endplate subsidence.

Accordingly, it is an objective of the present invention to provide an interbody implant that includes at least one surface that is compliant to the bone adjacent the compliant surface.

It is a further objective of the present invention to provide an interbody implant that includes oppositely positioned compliant surfaces that are positioned adjacent to the bone.

It is yet a further objective of the present invention to provide an interbody implant wherein the body of the implant is compliant to conform to the adjacent bone while still providing the spacing needed between the bones.

It is another objective of the present invention to provide an interbody spinal implant that includes a printed body including a plurality of spring bars integrally connected together to provide a predetermined compression rate.

Other objectives and advantages of this invention will become apparent from the following description taken in conjunction with the accompanying drawings wherein are set forth, by way of illustration and example, certain embodiments of this invention. The drawings constitute a part of this specification, include exemplary embodiments of the present invention, and illustrate various objects and features thereof.

### BRIEF DESCRIPTION OF THE FIGURES

FIG. 1 is a top, front, left perspective view of one embodiment of an interbody implant utilizing the bar spring support structure;

FIG. 2 is a top, front, left perspective view of one embodiment of an interbody implant utilizing the bar spring support structure;

FIG. 3 is a top, left, rear perspective view of another embodiment of an interbody implant constructed using bar springs;

FIG. 4A is a side view illustrating contralateral, caudal endplate subsidence;

FIG. 4B is a side view illustrating bilateral, caudal endplate subsidence;

FIG. 4C is a side view illustrating bilateral, caudal and cranial endplate subsidence;

FIG. 5 is an X-ray, as well as an illustration of the X-ray, showing a spine having a caudal endplate breach and sinking of the implant (cage) into the vertebral body;

FIG. 6 is an X-ray, as well as an illustration of the X-ray, showing a spine having a caudal endplate breach with antero-caudal implant tilt;

FIG. 7 illustrates an X-ray showing a spine having a caudal endplate breach and sinking of the implant (cage) into the vertebral body;

FIG. 8 illustrates a portion of a spine showing the direction of access to the disc space for various known types of spinal procedures; and

FIG. 9 is an illustration showing a portion of a human spine, illustrating the direction of access to the disc space for various known types of spinal procedures.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

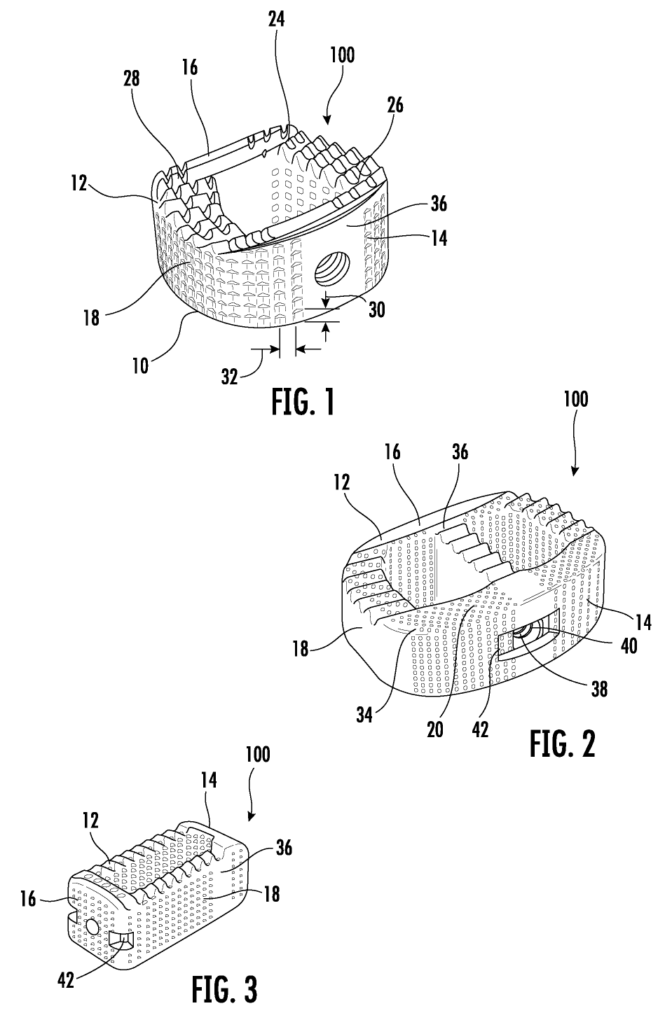

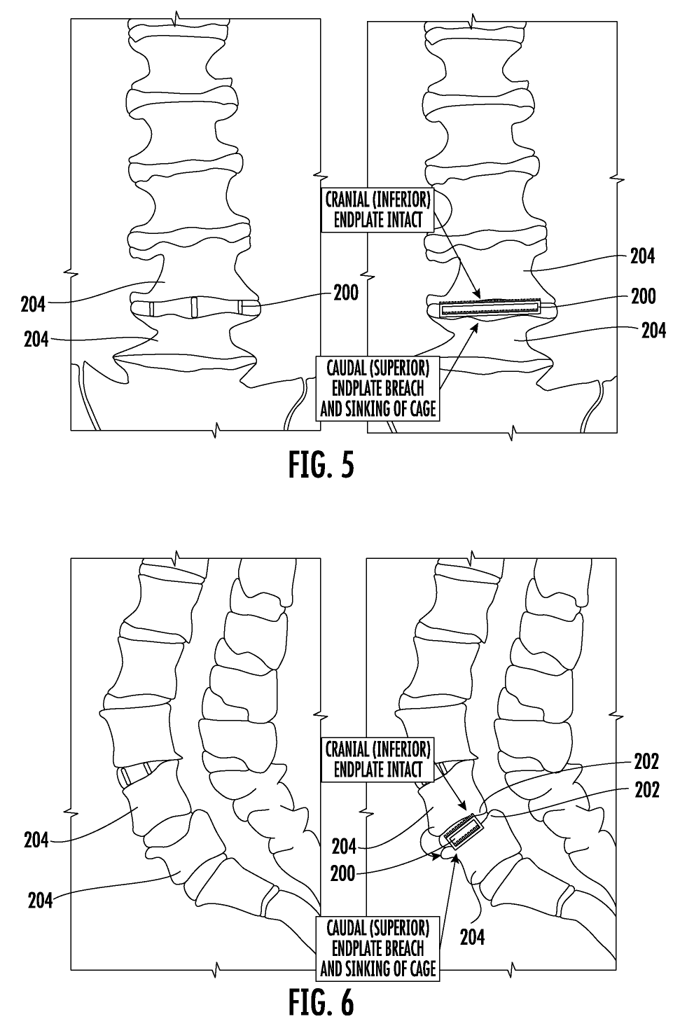

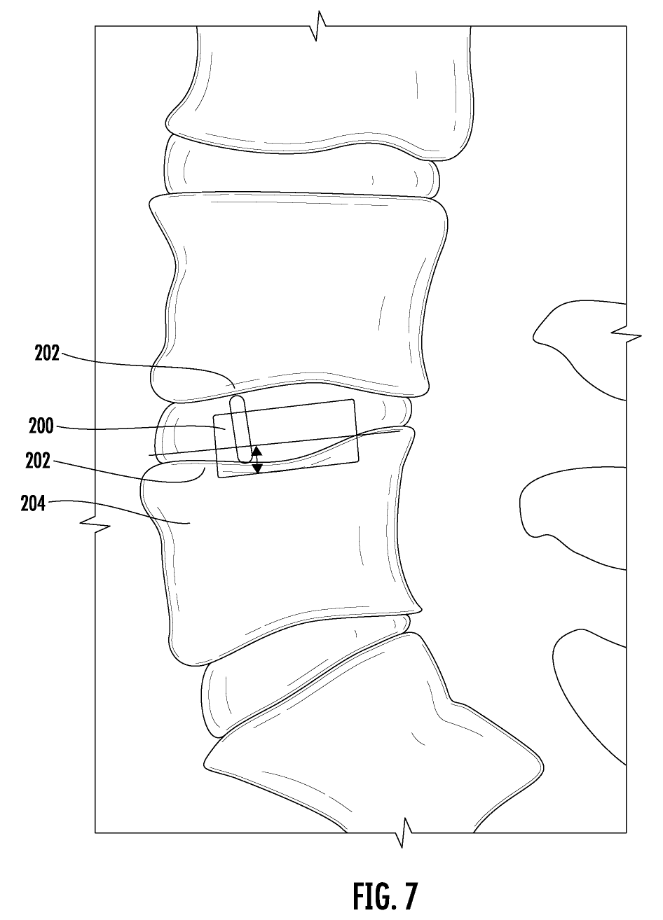

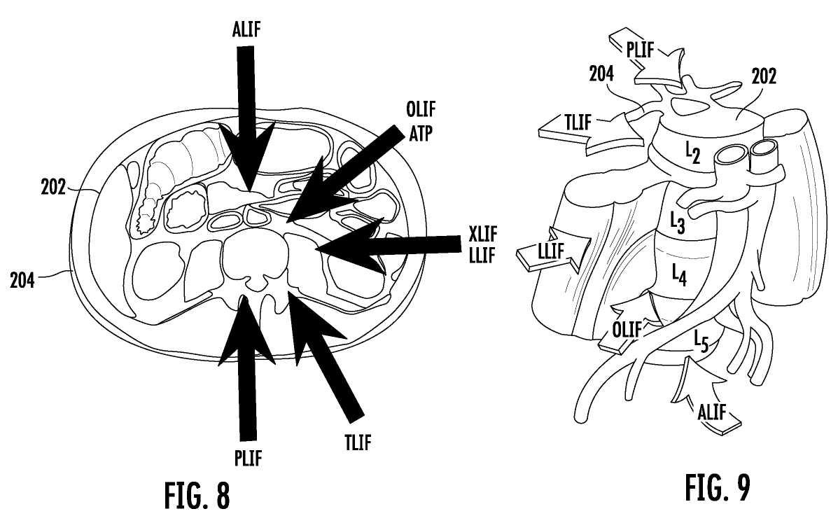

Referring generally to FIGS. 1-9, an interbody implant 100 for spinal corrective surgery is illustrated. The interbody implant 100 includes a bottom surface 10, a top surface 12, a front surface 14, a rear surface 16 and side surfaces 18. The surfaces are integral with respect to each other and are constructed and arranged to provide controlled compliance so that the surface contact area between the implant 100 and the bone surface is increased when compared to a hard surfaced implant. With respect to this disclosure, “compliance” is defined in the Oxford dictionary as “the property of a material of undergoing elastic deformation”. In at least one embodiment, at least the bone contacting surfaces, e.g. the top surface 12 and the bottom surface 10, include bar springs 20 formed to include an arch shape 24. The arch shaped 24 bar springs 20 may be secured at one or both ends 26 to a non-compliant solid portion 36 or to another layer of compliant bar springs 20. The uppermost surface 28 of the arches 26 provide a surface that can flex and comply with an adjacently positioned bone surface. Thus, each arch 24 has a predetermined spring rate that is determined by the connected or unconnected arch ends 26, the width 30 and thickness 32 of the bar spring 20, the radius of the uppermost surface 28 of the arch shape 24, and the temper and/or elasticity of the material forming the bar spring 20. It should be noted that the arched bar springs 20 may also be arranged in layers extending as far as all the way through the implant body. When extending more than one layer, the arched bar springs 20 may be connected to the adjacent layer at any point along the arch 24 to provide the desired spring rate. In a most preferred embodiment, the spring material is polyetheretherketone (PEEK), polyaryletherketone (PEAK) or polyetherimide (PEI) that has been printed in an inert atmosphere environment. However, it should be noted that printed metals, (titanium, stainless steel) may also be used without departing from the scope of the invention, as well as suitable combinations thereof. It should also be noted that while the bar springs 20 are illustrated as being square or rectangular when viewed from the end, other shapes may be utilized without departing from the scope of the invention. It should also be noted that while the bar springs are illustrated as being oriented so that the flat sides are the connecting sides, the bar springs may be oriented at various angles with respect to each other to provide different compliance characteristics.

Referring generally to the figures and more specifically to FIGS. 2 and 3, an embodiment of the interbody implant 100 includes the bar springs 20 arranged in a spaced apart layered arrangement, wherein each layer 34 extends in an angular relationship with respect to an adjacent layer 34. In at least one embodiment, the layers are arranged perpendicular or about perpendicular with respect to each other. In other embodiments, the layers may extend at various relationships from about five degrees to about ninety degrees with respect to each other without departing from the scope of the invention, the layers interconnected at the overlapping intersections. This construction allows the layers to flex with respect to each other, allowing compliance with the bone surface while still providing sufficient rigidity to support the bone structure. In some embodiments, the spacing and the angular relationship may change throughout the thickness of the implant 100. In this manner, the surface can be more compliant than the middle or central portion of the implant. Like the previous embodiments, the width 30 and thickness 32 of the bar springs 20 can also be altered to adjust the rate of compliance of the implant 100.

Still referring generally to the figures, some embodiments include solid formed sections 36. The solid formed sections 36 can be positioned at various positions around the implant to provide additional support, or for connection points 38 which may include threads or the like for attachment to insertion, positioning or removal tools. The connection points 38 also include keyways 42 or the like for rotation and directional control of the implant during insertion.

It should also be noted that the arched bar springs 20 and the flat bar springs 20 may be utilized in the same embodiment having the arched bar springs on the surface and the flat bar springs in the center or central portion of the implant.

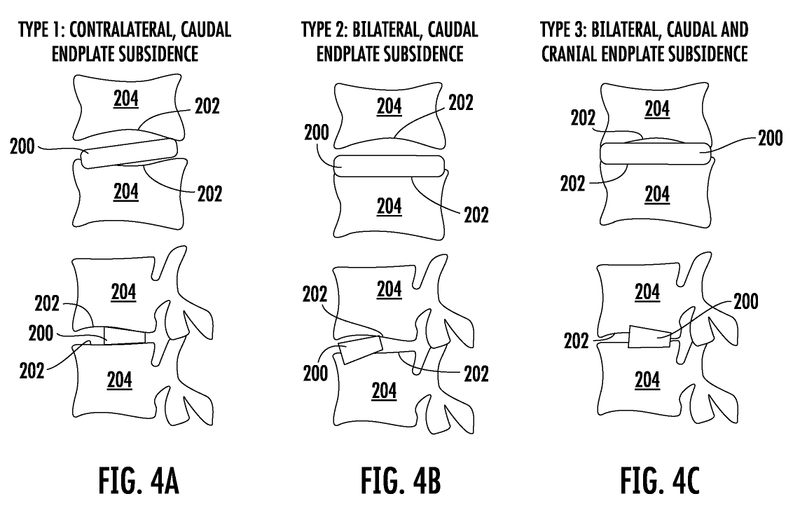

Referring to FIGS. 4-7, various illustrations are provided which illustrate the subsidence of a non-compliant interbody implant 200 into the endplate 202 of a vertebrae 204 after insertion between the bones. As stated above, the subsidence is often exacerbated or caused by the implant not contacting a sufficient amount of bone surface to support the load applied to the bone by the implant in use. This issue is exacerbated by several medical conditions, including osteoporosis, diabetes, age, sex etc. Therefore in some embodiments, the spring rate of the bar springs may be chosen by an algorithm that takes various bone factors into consideration and suggests a specific spring rate to comply with the bone of the patient to reduce the possibility of subsidence or cracking the bone. In some embodiments, a printer may be installed within or near the operating room wherein the implant is specifically constructed for a patient based upon bone inspection and testing or upon various factors relating to the patient wherein the springs are constructed specifically for the patient. It should also be noted that the present implant because of its porosity between the springs may be pre-packed or packed in the operating room to include bone growth proteins or bone powders, fragments or graft that enhance or increased bone growth around and through the implant.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

What is claimed is:

1. An interbody implant (100) comprising:
a bottom surface (10), a top surface (12), a front surface (14), a rear surface (16) and side surfaces (18), the surfaces formed integral with respect to each other and constructed and arranged to provide compliance so that the surface contact area between the implant (100) and a bone surface is increased when compared to a non-compliant implant.

2. The interbody implant of claim 1 wherein at least the bone contacting surfaces include a first layer of bar springs (20), each bar spring (20) is secured at one distal end (26) to a non-compliant structure, the uppermost surface (28) of the bar springs (20) providing a surface that can flex and comply with an adjacently positioned bone surface.

3. The interbody implant of claim 2 wherein the bar springs (20) are secured at both ends to the non-compliant structure.

4. The interbody implant of claim 2 wherein a second end of each bar spring (20) is secured to a compliant structure.

5. The interbody implant of claim 4 wherein the compliant structure is a second layer of bar springs (20), the second layer of bar springs (20) positioned adjacent to the first layer of bar springs (20).

6. The interbody implant of claim 1 wherein at least the bone contacting surfaces include bar springs (20), each bar spring (20) is secured at both distal ends (26) to a non-compliant structure, the uppermost surface (28) of the bar springs (20) providing a surface that can flex and comply with an adjacently positioned bone surface.

7. The interbody implant of claim 5 wherein both ends of each bar spring (20) are secured to a compliant structure.

8. The interbody implant of claim 6 wherein the compliant structure is a second layer of bar springs (20), the second layer of bar springs (20) positioned adjacent to the first layer of bar springs (20).

9. The interbody implant of claim 2 wherein each bar spring (20) is formed to include an arch shape (24).

10. The interbody implant of claim 9 wherein each bar spring (20) includes a predetermined spring rate.

11. The interbody implant of claim 2 including more than one layer of bar springs (20).

12. The interbody implant of claim 11 wherein the bar springs (20) are arranged in layers, one layer adjacent to the other.

13. The interbody implant of claim 12 wherein the distal ends of the first layer of bar springs (20) are connected to the bar springs (20) of a second layer of bar springs (20).

14. The interbody implant of claim 2 wherein the spring material is selected from the group consisting of polyetheretherketone (PEEK), polyaryletherketone (PEAK) or polyetherimide (PEI).

15. The interbody implant of claim 2 wherein the spring material is titanium.

16. The interbody implant of claim 12 wherein each layer is oriented in an angular relationship with respect to an adjacent layer to provide different compliance characteristics.
